(19) United States
(12) Patent Application Publication
(10) Pub. No.: US 2010/0168074 A1
(43) Pub. Date: Jul. 1, 2010

(54) Freezable Unit Dosage Delivery System and Method of Preparation

(75) Inventors: Christopher Culligan, Toronto (CA); Neil Smith, Halifax (CA); Rob Hart, Halifax (CA); Jay Sankey, Toronto (CA)

Correspondence Address: Sunstein Kann Murphy & Timbers LLP, 125 SUMMER STREET, BOSTON, MA, 02110-1618, US

(73) Assignee: Christopher Culligan, Toronto, ON (CA)

(21) Appl. No.: 12/595,808

(22) PCT Filed: Apr. 11, 2008
(86) PCT No.: PCT/CA2008/000682

§ 371 Date: Feb. 9, 2010

(30) Foreign Application Priority Data
Apr. 13, 2007 (US) 60911568

(51) Int. Cl. A61K 31/43; A61K 31/16; B65D 85/42

(52) U.S. Cl. 514/197; 514/629; 206/528; 53/476

(57) ABSTRACT

A freezable unit dosage delivery system, method of preparation, a solid unit dosage, method of treating symptoms and conditions, and kit is disclosed. The freezable unit dosage delivery system includes a composition, preferably containing one or more medicinal ingredients, having a freezing point less than 5° C.; a storage container comprising one or more cavity, each configured to receive a unit dosage quantity of the composition; and a sealing sheet for sealing engagement with the storage container. Upon freezing, the unit dosage quantity solidifies into a solid unit dosage that may be administered one of orally, rectally or vaginally for the local or systemic treatment of symptoms and conditions in a human.

### CROSS-REFERENCE TO RELATED APPLICATIONS

This application claims the benefit of U.S. Provisional Patent Application Ser. No. 60/911,568 filed Apr. 13, 2007, the contents of which are incorporated herein by reference in their entirety.

### FIELD OF THE INVENTION

The present invention relates generally to a freezable unit dosage delivery system for providing unit dosages suitable for oral, rectal or vaginal administration for locally or systemically treating symptoms or conditions.

### DESCRIPTION OF RELATED ART

Currently available throat lozenges or cough drops are generally in the form of tablet-size hard candies that are held in the oral cavity for long periods of time until dissolved. Unfortunately, these lozenges or cough drops often have rough or sharp edges that can further irritate or scrape the throat or mouth and the hard-candy nature of the lozenges result in slow dissolving of the lozenge. Although such lozenges can coat the oral cavity, they can only impart minimal relief from the dryness and general oral discomfort, for example, such as may be associated with the common cold or flu.

Current throat lozenges also suffer from other disadvantages. In certain groups, such as young children and the elderly, it can be difficult to hold the lozenge in their mouth for long periods of time or until the lozenge has completely dissolved and produced the desired local effect. Further, it can also be difficult for patients having painful mouth sores, lesions or other similar oral conditions to take long-lasting, hard oral lozenges because such medications can produce intolerable pain or discomfort.

Orally administered medications in solid dosage form, such as tablets and capsules, are generally intended to be swallowed. However, some individuals, including young children, demented persons and the elderly, may have a strong gag reflex, a physical impediment to swallowing or even a general aversion that presents difficulties in swallowing such medications. Oral administration may be particularly challenging where the dosage form is, or is perceived to be, large.

Certain orally-administered solid dosage forms are formulated so as to rapidly dissolve in the mouth. However, such tablets may suffer the disadvantage of being relatively fragile, subject to chipping or breaking upon removal from packaging or during handling for oral administration. Additionally, some individuals may also find medications in solid dosage form, or in liquids, unpalatable or otherwise generally unappealing, increasing the difficulty of administration.

Suppositories are generally inserted into a rectum or vagina where it dissolves and thus may be used to deliver local or systemic acting medicines, for example, analgesics or antibiotics. Certain individuals may feel discomfort associated with the use of a suppository, especially those that take a significant period of time to dissolve. For example, children, the elderly or the cognitively disabled may find the sensation of an inserted suppository disconcerting and attempt to dislodge it through movement before it is completely dissolved. Additionally, rectal suppositories may be expelled through defecation before the suppository is completely dissolved.

A need exists for a unit dosage delivery system and method for delivering an oral, rectal, vaginal, or other non-invasively delivered medication that may be used by a wide range of individuals, including young children, the cognitively disabled, persons suffering from temporary confusion or dementia, and the elderly, that addresses these and other disadvantages.

For example, a need exists for a unit dosage delivery system that may be administered in frozen form or that may be appealing to individuals including young children or that may mask unpalatable tastes or that may quickly and readily dissolve at body temperatures. Additionally, a need may also exist for a freezable medication delivery system that may be freezable in a conventional freezer or that may be of sufficient hardness to withstand removal from packaging without undue cracking or chipping or that may be readily prepared, stored and transported or that may be customized to an extent including as to dosage, formulation or configuration.

For oral medications in the nature of a throat lozenge, a need may exist for a medication delivery system that may also provide palliative relief for soreness or dry conditions in the mouth or may provide a cooling effect to throat swelling due to infection or trauma, or may impart a soothing effect beyond that produced by the carrier ingredients.

### SUMMARY OF THE INVENTION

A freezable unit delivery system for providing unit dosages in frozen form suitable for oral, vaginal or rectal administration to a human for locally or systemically treating symptoms or conditions, is disclosed.

In an aspect, the freezable unit dosage delivery system comprises a composition comprising at least one active ingredient and a physiologically or pharmaceutically acceptable carrier, the composition having a freezing point of 5° C. or less, a storage container comprising one or more liquid-impermeable cavities, each cavity configured to receive a unit dosage quantity of the composition; and a sealing sheet for sealing engagement with the storage container. The sealing sheet is adapted to cooperate with at least one cavity to define a unit dosage of the composition and to form a leak resistant seal enclosing the at least one cavity suitable for containing the unit dosage quantity of the composition within the cavity. The unit dosage quantity solidifies into a solid unit dosage of the composition within each cavity upon freezing.

In another aspect, a method for locally or systemically treating symptoms or conditions of a human in need of such treatment, is also disclosed. The method comprising the steps of: (a) providing a solid unit dosage prepared by: (i) providing a composition comprising at least one active ingredient and a physiologically or pharmaceutically acceptable carrier, said composition has a freezing point of about 5° C. or less; (ii) providing a storage container comprising one or more liquid-impermeable cavities, each cavity configured to receive a unit dosage quantity of the composition, (iii) placing a unit dosage quantity of the composition into at least one or more of the cavities; and (iv) applying a sealing sheet to sealing engage the storage container, the sealing sheet cooperating with at least one cavity to define a unit dosage of the composition and forming a leak resistant seal enclosing the unit dosage quantity of the composition within the cavity, (b) subjecting the storage container containing the unit dosages to freezing; (c) removing at least one of the solid unit dosages from said storage container; and (d) administering said at least one solid unit dosage to a human.

In another aspect, a method of preparing a freezable unit dosage delivery system is also disclosed. The method comprises the steps of: (a) providing a composition comprising at least one active ingredient and a physiologically or pharmaceutically acceptable carrier, said composition has a freezing point of about 5° C. or less; (b) providing a storage container comprising one or more liquid-impermeable cavities, each cavity configured to receive a unit dosage quantity of the composition; (c) placing a unit dosage quantity of the composition into at least one or more of the cavities; and (d) applying a sealing sheet to sealingly engage the storage container, the sealing sheet cooperating with at least one cavity to define a unit dosage of the composition and forming a leak resistant seal enclosing the unit dosage quantity of the composition within the cavity, wherein the unit dosage quantity solidifies into a solid unit dosage of the composition within each cavity upon freezing. In an embodiment, the method further comprises subjecting the storage container containing the unit dosages to freezing.

In another aspect, a kit comprising a freezable unit dosage delivery system is also disclosed. The kit comprises: (a) a composition comprising at least one active ingredient and a physiologically or pharmaceutically acceptable carrier, said composition having a freezing point of 5° C. or less; (b) a storage container comprising one or more liquid-impermeable cavities, each cavity configured to receive a unit dosage quantity of the composition; and (c) a sealing sheet for sealing engagement with the storage container, the sealing sheet adapted to cooperate with at least one cavity to define a unit dosage of the composition and to form a leak resistant seal enclosing the unit dosage quantity of the composition within the cavity; and (d) an instruction to subject the storage container containing the unit dosages in at least one or more of the cavities to freezing. The unit dosage quantity solidifies into a solid unit dosage of the composition within each cavity upon freezing. The solid unit dosage form is suitable for one of oral, rectal or vaginal administration.

In another aspect, a solid unit dosage for one of oral, vaginal or rectal administration for locally or systemically treating symptoms or conditions of a human in need of such treatment, comprising a unit dosage quantity of a composition, the composition comprising at least one active ingredient and a physiologically or pharmaceutically acceptable carrier, and having a freezing point of 5° C. or less. The unit dosage quantity is in liquid or flowable form at room temperature prior to freezing, and the unit dosage quantity solidifying into a solid unit dosage upon freezing.

Preferably, the unit dosage quantity is in a liquid or flowable form at room temperature, for example, when between 10° C. to 35° C. prior to freezing, and the unit dosage form freezes at a temperature below 5° C., or below 0° C., for example, between −20° C. and 0° C.

Preferably, the unit dosage quantities in liquid form is shaped by the cavities of the storage container upon freezing into a configuration that is suitable for one of oral, rectal or vaginal administration. The solid unit dosages may be configured into the form of a lozenge, tablet, capsule, ring, star, geometric shapes such a cubes, rhomboids, cylinders or spheres, suppositories, torpedoes or other novelty shapes such as candy confectionaries, for example, jelly beans, figures, dinosaurs, or the like.

Preferably, the composition comprise one or more active ingredients together with a physiologically acceptable carrier, including a pharmaceutically acceptable carrier. Preferably, the carrier comprises a solvent that freezes below 5° C., including below about 0° C. The solvent used may include, but is not limited to, water, an alcohol, a polyol, a polyether polyol, a derivative of a polyether polyol, glycerol, gelatin, mineral oil, olive oil, corn oil or any other consumable biologic or synthetic oil. The active ingredients may be dissolved, suspended, dispersed, or otherwise mixed in the carrier. For example, the composition may be in liquid or otherwise flowable form at room temperature with the active ingredients dissolved therein or may be in a gelatin format with the active ingredient suspended in a gelatin-like suspension.

In another embodiment, the active ingredient includes an herbal, nutritional or pharmaceutical active ingredient, or combinations thereof. The active ingredient may include, but is not limited to, antibiotic agents, antifungal agents, anti-infective agents, antiviral agents, analgesics, decongestants, antihistamine, sedatives, anxiolytics, diuretics, stimulants, expectorants, muscle relaxants, antacids, anti-inflammatories, antipsychotics, antidepressants, neuroleptics, antipyretics, antitussives, steroids, chemotherapies, antiarrhythmics, diuretics, antinausea, hyperglycemics, antiepileptics, motility agents, antihypertensives or the like, herbal medicines, anti-oxidants, vitamins and minerals, or the like.

Symptoms or conditions that may be treated include, but is not limited to, bacterial, viral, and fungal infections, including a common cold or flu and the symptoms associated therewith such as nasal and sinus congestion, sore throat and cough, bronchitis, upper respiratory tract infection, pneumonia, aches, pain, headache, hoarseness, oral or rectal discomfort and irritation, malaise, fatigue, insomnia, mood disorders, depression, cholesterol level disorders, allergic reactions, stress, sleeplessness, anxiety, digestive disorders, gastrointestinal disorders, kidney disorders, bowel and urinary disorders, dementia, delirium, fever, cough, hypertension, heart and vascular disorders, diabetes and related conditions, inflammation, autoimmune and immunodeficiency disorders, respiratory disorders, and the like, including conditions and symptoms associated with the elderly, aging, and the young, as well as less typical conditions and symptoms, including those due to or associated with injury, illness or rare disorders. As will be readily appreciated, this listing is merely exemplary and not exhaustive, intending to illustrate the variety of diseases, disorders, conditions, symptoms, and the like, that may be treated.

In an embodiment, a storage container, such as a blister pack, comprising at least one unit dosage or a plurality of unit dosages, containing a freezable composition with an antibiotic, for example, amoxicillin as the active ingredient for treating an infection.

The composition may also include flavouring and colour. The composition may also include buffering agents, stabilizers, surfactants, preservatives and sweeteners.

The storage container containing the unit dosage may be pre-packaged, transported, and stored at room temperature, to be frozen prior to administration. The unit dosage form is generally in frozen form at the time of oral, rectal or vaginal administration.

In another embodiment, the freezable composition includes acetaminophen. In yet another embodiment, the freezable composition includes ginseng or Echinacea.

In an embodiment, a unit dose quantity of a composition containing an active ingredient is added to each cavity of a storage container, such as a blister pack, which is then sealed, for example, by heat sealing, adhesives, lamination or using mechanical means. In an embodiment, the mechanical means may be applied by a person manually, rather than mechanically, to seal the sealing sheet over a cavity.

The storage container may be accompanied by instructions to subject the storage container containing the unit dosages to freezing, for example, to a temperature of below about 5° C., preferably below 0° C., for a period of time sufficient to freeze the unit dosages into solid form.

In another embodiment, a storage container, such as a blister pack, comprises at least one frozen lozenge or a plurality of oral frozen lozenges for treating symptoms associated with the common cold or flu. The frozen lozenge can be sealed within the storage container. The frozen lozenge being a frozen aqueous, or other non aqueous solution, and further comprising an herbal ingredient, a flavoring agent and water.

As used herein, the term “treating symptoms or conditions” or “treating” or similar terminology includes, but is not limited to, preventing and ameliorating symptoms and conditions associated with dysfunction, distress, injury, disease or disorders, particularly to persons experiencing or at risk of such dysfunction, distress, injury, disease or disorder.

### BRIEF DESCRIPTION OF THE DRAWINGS

FIG. 1 shows diagrammatically a top view of a storage container in the form of a blister pack for holding frozen unit dosages of a composition of the present invention.

FIG. 2 shows diagrammatically a cross-sectional view of a storage container having cavities for holding unit dosages of a frozen composition of the present invention.

FIG. 3 similarly shows diagrammatically a cross-sectional view of a storage container having cavities for holding freezable unit dosages of a composition of the present invention.

FIG. 4 is a perspective view of a single unit dosage separated from a storage container and further illustrating a method of access to the unit dosage.

### DETAILED DESCRIPTION OF PREFERRED EMBODIMENTS OF THE INVENTION

In the description that follows, when a preferred range, such as 5 to 25 (or 5-25), is given, this means preferably at least 5 and, separately and independently, preferably not more than 25.

The compositions of the present invention are useful for locally or systemically treating symptoms or conditions. The composition may comprise one or more active ingredients together with a physiologically acceptable carrier, including a pharmaceutically acceptable carrier. The active ingredients includes herbal and/or pharmaceutical active ingredients, which may include, but is not limited to, antibiotic agents, antifungal agents, antiinfective agents, antiviral agents, analgesics, decongestants, antihistamine, sedatives, anxiolytics, diuretics, stimulants, expectorants, muscle relaxants, antacids, anti-inflammatories, antipsychotics, antidepressants, neuroleptics, antipyretics, antitussives, steroids, chemotherapies, antiarrhythmics, diuretics, antinausea, hyperglycemics, antiepileptics, motility agents, antihypertensives or the like. As will be appreciated, this listing is merely exemplary and not exhaustive, intending to illustrate the variety of active ingredients that may be used for the treatment of diseases, disorders, conditions, symptoms, and the like.

Symptoms or conditions that may be treated include, but is not limited to, bacterial, viral, and fungal infections, including a common cold or flu and the symptoms associated therewith such as nasal and sinus congestion, sore throat and cough, bronchitis, upper respiratory tract infection, pneumonia, aches pain, headache, hoarseness, oral or rectal discomfort and irritation, malaise, fatigue, insomnia, mood disorders, depression, cholesterol level disorders, allergic reactions, stress, sleeplessness, anxiety, digestive disorders, gastrointestinal disorders, kidney disorders, bowel and urinary disorders, dementia, delirium, fever, cough, hypertension, heart and vascular disorders, diabetes and related conditions, inflammation, autoimmune and immunodeficiency disorders, respiratory disorders, and the like, including conditions and symptoms associated with the elderly, aging, and the young, as well as less typical conditions and symptoms, including those due to or associated with injury, illness or rare disorders. As set out aforesaid, this listing is merely exemplary and not exhaustive, intending to illustrate the variety of diseases, disorders, conditions, symptoms, and the like, that may be treated.

As will be readily appreciated by those skilled in the art, the present invention contemplates that a wide range of active ingredients may be incorporated into the freezable composition, selected for the treatment of a wide variety of conditions and symptoms.

For example, influenza (i.e., the flu), common cold, pneumonia, bronchitis or upper respiratory tract infections such as Streptococcus pneumoniae and Hemophilus influenzae are conditions that may be treated. Symptoms associated with such conditions can comprise, but are not limited to, nasal drainage, nasal congestion, sinus congestions, headache, fever, myalgia, sneezing, sore throat, scratchy throat, oral irritation, oral discomfort, oral pain, cough, hoarseness, or combinations thereof and the like. Such symptoms can cause irritation, itchiness, soreness and general discomfort in the oral cavity and/or throat area of a human.

The compositions of the present invention, particularly in solid unit dosage form, may be administered one of orally, rectally or vaginally. The compositions may have local or systemic effect, for example, through absorption through mucous membranes into the bloodstream or ingestion of the still frozen or melted solvent and solutes into the stomach for typical gastrointestinal absorption.

The compositions, or a unit dosage quantity thereof, preferably freeze and become solid below 5° C., more preferably below 3° C., more preferably below 0° C., although freezing may occur in temperature ranges between 0° C. to −20° C. The compositions are preferably in liquid or flowable form at room temperature, between about 10° C. to 35° C., preferably between 15° C. to 25° C. before freezing. It is to be appreciated that it is the composition that is substantially in liquid or flowable form. Constituents, for example, active ingredients, may be present in a non-liquid form and, for example, be suspended in a flowable medium.

The compositions, or a unit dosage quantity thereof, can be frozen by subjecting them to low temperatures, such as below 5° to 0° C., or lower, for example, between −20° C. to 0° C., for a period of time sufficient to freeze and convert the flowable composition, particularly, a unit dosage quantity thereof, into a frozen solid. Preferably, the compositions can be placed in a freezer or the freezer section of a refrigerator in order to freeze the unit dosages. In the frozen, solid state, a unit dosage of the compositions can be orally, rectally or vaginally administered as the case may be, to treat or provide relief from the symptoms and conditions described above and herein. When placed in the oral, rectal or vaginal cavity as the case may be, the frozen, solid unit dosage of the composition quickly melts. The melted unit dosage of the composition may contact, cover or coat the cavity locally, or otherwise be absorbed into the bloodstream or through the gastrointestinal system.

As will be readily appreciated, by “frozen solid” or “frozen” or similar terminology, the composition, or a unit dosage quantity thereof, in its frozen state is one where the unit dosage is of sufficiently solid form so as to be handled for oral, rectal or vaginal administration (as the case may be). The frozen composition, particularly, in a unit dosage quantity thereof, does not have to be completely frozen provided that a substantial portion of the unit dosage of the composition, preferably containing a substantial portion of the active ingredient, is of sufficiently solid form so as to be capable of oral, rectal or vaginal administration.

For example, in the oral cavity, the melted or thawed unit dosage of composition may contact, cover or coat the tongue, throat and inner cheek surfaces. The frozen composition unit dosage quickly becomes smooth as a result of the melting action and therefore does not scratch or otherwise irritate the oral cavity or throat area. In some cases, the frozen unit dosage may assist in masking an unpalatable taste. In other cases, where an individual experiences throat swelling due to, for example, infection or trauma, the frozen unit dosage may impart a cooling or numbing effect. As the oral cavity and throat area become coated with the melted composition, the ingredients of the composition can absorb into the consumer's blood stream through the oral mucosal and sub-lingual membranes. The composition can continue to be absorbed into the bloodstream of the consumer. Similar activity may occur vaginally or rectally.

The frozen compositions described herein can be in a unit dosage, such as a lozenge which can comprise a cough drop, or a suppository. The unit dosage may contain a selected amount of active ingredient. By “active ingredient” both herbal, pharmaceutical or other medicinal ingredients having therapeutic, prophylactic or ameliorative effect on symptoms or conditions described aforesaid, are contemplated. By “medicinal” or “medication”, substances that may be used to treat, prevent, suppress, ameliorate, and improve conditions and symptoms that may be experienced or suffered by a human, including herbal, pharmaceutical including over-the-counter, and nutritional including supplements, active ingredients are contemplated. The selected amount of active ingredient will vary depending upon the nature, location and severity of the condition or symptom to be treated, prevented or ameliorated, the nature of the composition and its constituent components, and also upon the route of administration. As will be appreciated, the dose and dose frequency will vary according to the age, physical condition, size, body mass, response, and similar factors, of the consumer or patient.

Preferably, the weight of a unit dosage is in the range of 3 to 20 grams. Preferably, the weight is in the range of 3.5 to 5 grams for an oral, rectal or vaginal lozenge. As will be appreciated, the actual weight may vary depending on a number of factors, including the desired configuration or shape in frozen form, ingredients, constituents and mode of administration.

The dosage forms of the present invention may comprise the administration of a frozen composition in a single unit dose, a double unit dose, or more than two unit doses, or a fractional unit dose to be taken once, twice, three times, four times, 6 times, or other frequency, during a 24-hour period of time.

The composition is generally packaged in a storage container having, preferably, a plurality of individual cavities that contain individual unit dosages. The composition, which includes the carrier, active ingredient(s), and other constituents, in liquid or flowable form, is added to the cavities. Preferably, the amounts of composition added constitute a unit dosage quantity. For a medication, including a pharmaceutical, nutritional or herbal medication, the amount of composition added includes an appropriate quantity of active ingredient sufficient to constitute a dose. However, it can be appreciated that more than one unit dose may be required to be administered to constitute a sufficient dose for a selected patient or consumer.

The cavities of the storage container are adapted to receive and contain the unit dosage composition in flowable form when in room temperature prior to freezing, and is thus liquid impermeable. By “liquid impermeable”, the material is capable of retaining the composition in liquid or flowable form without leakage, in other words, is leak resistant. By “room temperature”, this would include a temperature range of 10° C. to 35° C., preferably 15° C. to 30° C., and more preferably, 15° C. to 25° C., prior to freezing. A liquid impermeable sealing sheet encloses the cavities so as to form individual compartments, each containing a unit dosage of the composition, including in liquid or flowable form at room temperature, prior to freezing.

The storage container is adapted to be freezable such that the liquid or flowable unit dosage of the composition may freeze and solidify, generally taking the shape of the cavity within which the unit dosage is contained. Preferably, the sealing sheet is releaseably sealable or ruptureable to facilitate the removal of a frozen unit dosage from the storage container.

The composition, in a predetermined formulation, for example, including an active ingredient present in a predetermined concentration, and in predetermined unit dosage quantities, may be added to the cavities of the storage container by a manufacturer. The storage container may be preselected to contain a predetermined number of cavities, each of which is filled with a unit dosage quantity. The unit dosage quantities may be securely sealed within the cavities of the storage container. Such packaged storage containers containing preformulated and predetermined quantities of composition in unit dosages may be packaged in a form suitable for transport and storage. The packaged storage containers containing unit dosages may be frozen prior to transport and delivered to its destination in a frozen form. Alternatively, the packaged storage containers containing unit dosages may be packaged in liquid or flowable form, together with instructions, to subject the unit dosages individually or in packaged form, to freezing, which may include particulars as to a suitable temperature and a sufficient period of time to freeze the unit dosages into solid form prior to administration to a patient or a consumer. Individual unit dosages, or groups thereof, in frozen form may be separated from or otherwise removed from the storage container for administration thereof.

Alternatively, the composition may be a predetermined formulation, for example, including an active ingredient, which is added to the cavities of the storage container in unit dosage quantities by, for example, a pharmacist or dispensing chemist. Alternatively, the composition may be customized, for example, prepared by the pharmacist or chemist in accordance with directions from a physician or dentist, including as to the constituents, concentration and unit dose quantities. Such a composition may, for example, comprise as constituents two or more active ingredients, which are dissolved or suspended, or both, in a specified solvent. The pharmacist or chemist may use pre-existing constituent ingredients, for example, an antibiotic available in powder form mixed with distilled water. Additional ingredients may be added, for example, stabilizers, buffers, flavouring or sweeteners.

The storage container may be available comprising a predetermined number of cavities and in a predetermined configuration, for example, in a matrix comprising three rows and seven columns. Such a storage container may be used, for example, for containing three unit dosages to be taken three times a day for seven days. Alternatively, the storage container may be available, for example, in sheet form comprising a larger number of cavities, for example, in sheets comprising thirty rows by thirty columns. The storage container may comprise a selected number of cavities, for example, as may be selected by the pharmacist or chemist depending on the number of unit dosages to be dispensed. Such storage containers in sheet form may include perforations or weakened portions between cavities to facilitate separation of a desired number of cavities to be filled with unit dosage quantities of composition. As will be appreciated, the size of the sheet and configuration of the cavities may vary significantly, taking into account factors including the size and shapes of the cavities, the number of unit dosages to be made available, transportability and storage considerations, and the like.

The size, shape and general configuration (collectively “configuration”) of a frozen unit dosage is generally defined by the configuration of the cavity. Particular configurations may be suited, and thus, selected for particular purposes, for example, for ease of administration, storability, handling, or even aesthetics or playfulness. Thus, a variety of storage containers having a variety of cavity configurations may be available to, for example, a pharmacist or chemist who may select a particular storage container for its specific cavity configuration to suit a particular purpose. As will be appreciated, such storage containers may also be prefabricated with a variety of cavity configurations and prefilled with unit dosages of a specific formulation composition by a manufacturer, thereby providing the ultimate consumer or a patient with a choice of a variety of configurations for a given formulation. Such storage containers may be shipped with preformulated and prepackaged unit dosage quantities already frozen or in liquid or flowable form for subsequent freezing.

Additional solute may also be added by a pharmacist or a chemist in order to fill a cavity to ensure that, following freezing, each frozen unit dosage is substantially uniform in size, including regardless of the amount of active ingredient contained within the unit dosage quantity of the composition.

Such storage containers may be separately or attachedly provided with a liquid impermeable sealing sheet. The sealing sheet is adapted to securely seal liquid or flowable unit dosages within the cavities of the storage container and to be leakage resistant. Such a sealing sheet may be applied by the pharmacist or chemist following the deposit of unit dosage quantities of composition into the cavities. A variety of means may be employed to effect such seal. For example, the sealing sheet may include a discontinuous adhesive to enable sealable adherence of the sealing sheet to a film sheet of the storage container with the use of pressure where the adhesive does not come into contact with the unit dosage quantities of composition contained within the cavities.

Alternatively, the sealing sheet may be provided with a mechanical sealing mechanism, for example, leak resistant interlocking sealing ribs with a first profile provided on the storage container at or about the perimeter defining a cavity opening and a second corresponding interlocking profile provided on the sealing sheet. Upon application of pressure, the two interlocking profiles engage and create a leak resistant seal maintaining the liquid or flowable unit dosage within the cavity. As another example, the sealing sheet may be provided with a protruding ridged “tongue” profile that is sized and shaped to sealingly engage a corresponding recessed “groove” profile provided surround the outer perimeter of each cavity. Upon manual application of pressure, the protruding ridged tongue profile on the sealing sheet frictionally engages the corresponding recessed “groove” profile for creating a leak resistant seal in tongue-in-groove engagement for maintaining the liquid or flowable unit dosage within a cavity. It can be appreciated that these mechanisms may be varied, for example, by construction, shape of profiles, composition, coating materials, and the like, to improve upon the stability, strength and leak resistance of the seal. The materials used may also be treated so as to improve leak resistance. Additional gasket material may also be provided to improve leak resistance.

As will be appreciated, the layout and construction of such seals may vary. For example, each cavity opening in a storage container may be individually encircled about its perimeter to which individual sealing sheets with corresponding interlocking or tongue profiles may be applied. Alternatively, for a storage container comprising a row of cavities or a matrix of cavities in linear arrangement, a regular pattern of a profile for example, squares, circles, rectangles, triangles, or the like, may be provided about the perimeter defining each cavity opening. Sealing sheets configured into one or more rows and provided with corresponding sealing profiles, may be applied for sealing engagement with a corresponding row on the storage container. For example, a storage container with a linear arrangement of three rows and seven columns of cavities may be provided with a regular pattern of rectangular grooved profiles, each encircling a perimeter of a cavity. Sealing sheets sized to rectangularly encircle the perimeter of one cavity on a storage container and having a corresponding tongue profile are individually applied to sealingly engage each cavity. Alternatively, three sealing sheets, each comprising a row of seven rectangular tongue profiles, each sized, configured and positioned to correspondingly engage the grooved profiles encircling one row of cavities on a storage container, are each applied to one row of the storage container.

As a further alternative, a storage container may be formed with a lipped perimeter region surrounding the cavity openings. A frame attached to or separate from the storage container may be sized and configured to securely frictionally engage the perimeter lip of one or each of a plurality of cavities. A sealing sheet may comprise a flexible liquid impermeable sheet, such as a polymeric or cellulosic web, and is sized to broadly cover one or a plurality of cavities. The sealing sheet may be placed over one or a plurality of cavities. The frame may then be placed over the sealing sheet, and with manual pressure, be caused to frictionally engage the perimeter lips surrounding the cavities to be covered, thereby forming a leak resistant seal over the cavities. Alternatively, the sealing sheet may comprise a plurality of lids, each of which individually cover each cavity.

The storage container and sealing means may be manufactured using disposable material, intended for one time use, or alternatively, may be manufactured from more sturdy material, enabling reuse, for example, for refilling a prescription. In an example, a sealing sheet may comprise a plurality of individual lid means, for example, linearly arranged, each lid means hingely attached to the storage container and positioned to cover a corresponding plurality of cavities linearly arranged. A composition may be formulated by a pharmacist, and a unit dosage quantity placed into each cavity, for example, in the form a cubic compartment, and subsequently frozen.

As will also be appreciated, perforations, sheet weakening or the like may be provided so as to facilitate the separation or removal of one or more unit dosages. Such perforations or sheet weakenings are preferably positioned so as to not derogate or otherwise interfere with effectiveness of a leak resistant seal. As will also be appreciated, this description of mechanical sealing means is intended to be illustrative and not exhaustive, of other suitable sealing means that may be employed.

Such packaged storage containers containing unit dosages quantities of composition in liquid or flowable form at room temperature may be dispensed to a patient or consumer together with instructions, to subject the unit dosages individually or in packaged form, to freezing which may include additional instructions as to suitable temperatures and a sufficient period of time, to freeze the unit dosages into solid form prior to administration to a patient or a consumer. Individual unit dosage, or groups thereof, in frozen form may be separated from or otherwise removed from the storage container for administration thereof.

The frozen compositions of the present invention provide an oral, rectal or vaginal unit dosage delivery system that may be tolerated well by a wide variety of consumers.

For consumers suffering from a local oral or systemic condition, such as the common cold or influenza, the solid, frozen compositions of the present invention provide a prolonged cool, soothing feeling to the oral cavity and throat area. For instance, the frozen compositions can treat or suppress symptoms such as sore throat, scratchy throat, hoarseness, cough, oral irritation, oral discomfort or oral pain by providing a cool, soothing oral dosage, such as in the form of a lozenge, against the areas of the oral cavity experiencing such symptoms. The cooling or soothing benefit of the frozen compositions can be combined with an herbal ingredient or ingredients contained in the composition that effectively treat or suppress the symptoms of the consumer, such as nasal drainage, nasal congestion, sinus congestions, headache, fever, myalgia, sneezing, sore throat, scratchy throat, oral irritation, oral discomfort, oral pain, cough or hoarseness. The frozen composition further provides wetness and general hydration to the oral cavity and relieves the dry feeling that is associated with symptoms such as sore throat.

For pediatric consumers or young children, disabled and the elderly, who often resist taking oral medications, the frozen compositions of the present invention may provide a safe and pleasing way for orally administering a medication. If these consumers were to prematurely swallow and choke on the frozen unit dosage, unlike hard candy lozenges or cough drops, the frozen composition may quickly melt and the likelihood that harm would be caused is substantially reduced. A frozen unit dosage may further assist in masking an unpalatable taste due to a constituent in the composition, and, for example, by simulating the shape and texture of a candy confectionary or even ice chips, may appeal to a child or a confused individual.

Where a frozen unit dosage is sized and shaped, for example, comparable to candy or cough drops, the frozen unit dosage may rapidly melt thereby increasing the likelihood that an appropriate dose is administered. Further, in frozen form, there is an increased likelihood that a unit dosage may be freed or removed from its packaging and administered without disintegrating, breaking, cracking or spilling.

Similarly, in the case of a frozen unit dosage for rectal or vaginal use, for example, in the form of a suppository containing an antibiotic, the frozen unit dosage may quickly melt upon administration, thereby coating and wetting the area surrounding the administration site, providing, for example, soothing, cooling relief and a degree of lubrication, while melting relatively rapidly to remove the sensation of the inserted unit dose and releasing medication for systemic or local administration, as the case may be. In some cases, the frozen unit dosage may provide a desensitizing or numbing effect to reducing discomfort.

As another example, antipyretics (such as acetaminophen) are a valuable therapy for children and confused individuals, as much as the general population when suffering from increased body temperature. It is well known that fever leads to confusion and increased diaphoresis (sweating) that leads to a reduction in electrolytes that causes dehydration, a deterrent to recovery. The said delivery device would reduce the difficulties of delivering these antipyretics to the populations that most often resist taking the typical oral liquid or tablet forms by placing them in a more desirable frozen alternative.

The present invention will now be described with reference to the drawings, wherein like reference numerals are used to refer to similar elements throughout. It is to be appreciated that the various drawings are not necessarily drawn to scale from one figure to another nor inside a given figure, and in particular that the size of the components are arbitrarily drawn for facilitating the understanding of the drawings. In the following description, for purposes of explanation, numerous specific details of preferred embodiments are set forth. However, the present invention can be practiced without requiring all of these specific details. Additionally, other embodiments of the invention are possible and the invention is capable of being practiced and carried out in ways other than as described. The terminology and phraseology used in describing the invention is employed for the purpose of promoting an understanding of the invention and should not be taken as limiting.

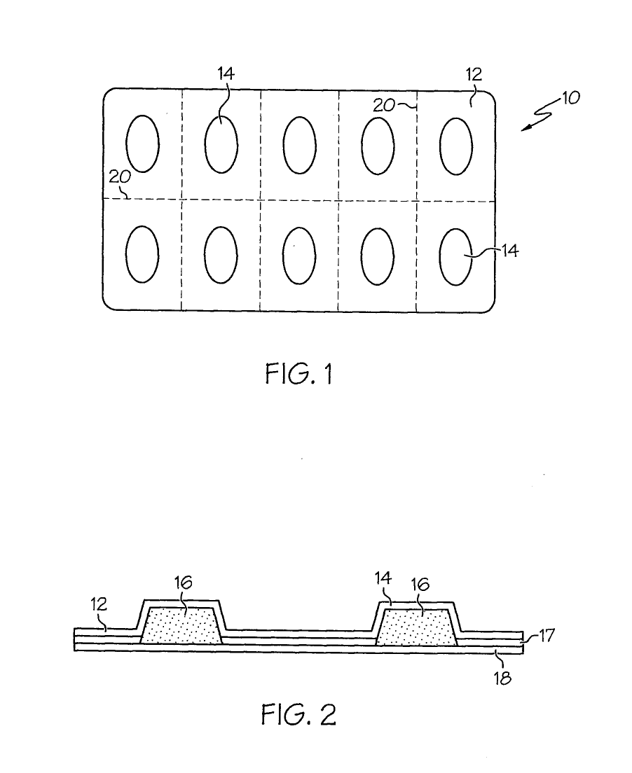

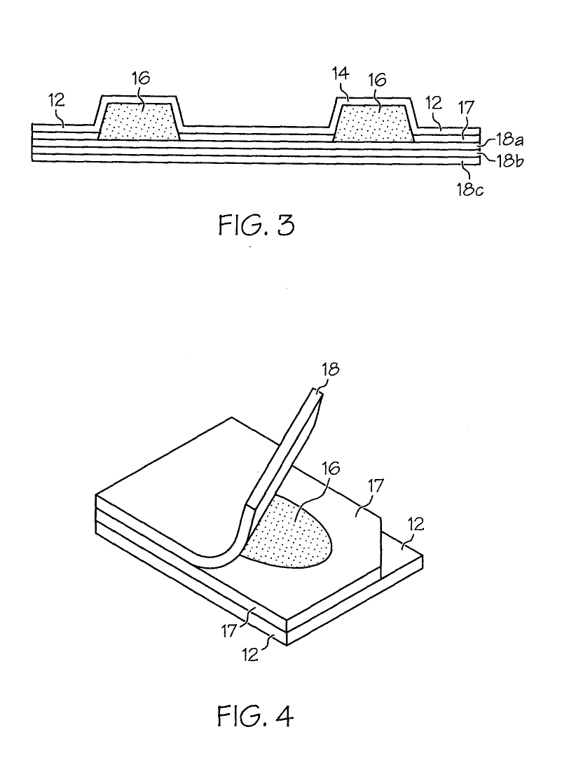

In one embodiment, the storage container 10, as seen in FIG. 1, can be a blister pack, similar to those known in the art. Examples of embodiments of materials employable in blister packages and methods of making the same are set forth in U.S. Pat. Nos. 3,905,479; 3,912,082; 3,924,747; 3,835,995; 3,912,081; 3,924,746; 3,809,220; 3,809,221; 3,811,564; 3,872,970; 3,899,080; 3,921,805; 3,941,248; 5,046,618 and 7,000,769. FIG. 1, illustrates a method of preparing the compositions of the present invention for oral administration in a lozenge form comprises packaging the compositions in a storage container 10, such as a blister pack having a releasably-sealable or ruptureable film, that is suitable for freezing. Preferably, the film is substantially liquid impermeable. As shown, the storage container 10 comprises a liquid-impermeable film sheet 12 having a plurality of depressions or cavities 14 extending from the plane of said film sheet 12. The film sheet 12 can be made from any variety of translucent, transparent or opaque plastics, for example, polyvinyl chloride, polyvinyl dichloride, polyvinylidene chloride, polypropylene, polyethylene, polychlorotrifluoroethylene, and combinations thereof. As shown in FIGS. 2 and 3, each cavity 14 of the film sheet 12 preferably holds a unit dosage 16, which can be composed of a composition of the present invention.

The cavities 14 of a film sheet 12 or storage container 10 can be arranged in numerous configurations. As an example, in one embodiment, a film sheet 12 can comprise at least one ordered arrangement (i.e., row or column) of cavities 14. For example, a film sheet 12 can comprise at least one or two rows or columns of cavities 14. In another example, a film sheet 12 can comprise four rows or columns of cavities 14. A storage container 10 can hold a single unit dosage 16, or a number of unit dosages 16, for example, 1-100, 1-50, 1-40, 1-30 or about 2, 4, 6, 8, 10, 12, 16, 18, 20, 24, 36 or 48 unit dosages 16. Depending on the number of unit dosages 16 contained in a storage container 10, the storage container 10 can comprise dosages for a period ranging from about one day to about two weeks, or longer. Thus, a storage container 10 can be used to assist a consumer in conforming to a dosing or medication regime by having visual indicia, such as labeling, that indicates “a.m.” and/or “p.m.” or days of the week along the rows or columns of cavities 14. Alternatively, for unit dosages that may be administered on an as-need basis, for example, acetaminophen administered for the temporary relief of pain, the storage container 10 may present unit dosages in amounts not dependent upon a particular dosage regimen.

The shape of the cavities 14 of a film sheet 12 or storage container 10 may be selected such that, upon freezing, the unit dosage quantity of the composition assumes a configuration that would facilitate administration. For oral administration, the shape of the cavities 14 may be selected to be spherical or other geometric shape, tablet, or capsule shape, for example, to facilitate oral administration, or even novelty shapes such as stars, rings or diamonds, animals or figures, to appeal to children. For rectal or vaginal administration, the shape of the cavities 14 may be selected to be torpedo or the like, to facilitate insertion. As will be appreciated, the shape of the cavities 14 of the film sheet 12 or storage container 10 may be fabricated to assume a wide variety of shapes, sizes and configurations.

The unit dosages 16 are retained and enclosed within the cavities 14 of a film sheet 12 by a sealing sheet 18. The sealing sheet 18 can be attached to the plane of the film sheet 12 by various techniques, for example, laminating, heat sealing or with an adhesive. As shown in FIGS. 2 and 3, an adhesive layer 17 can be positioned between the film sheet 12 and sealing sheet 18. Although the use of an adhesive layer 17 is optional, it can promote adhesion and prevent delamination of the sealing sheet 18 from the film sheet 12, which can be caused from normal use and wear and tear on a storage container 10. Bonding of the sealing sheet 18 to the film sheet 12 occurs such that the cavities 14 are sealed, with selected portions, away from the cavities 14, left unsealed 15.

As set out aforesaid, in circumstances where a preformulated composition is not prepackaged into unit dosages by a manufacturer, for example, where a pharmacist or chemist prepares a storage container with a liquid or flowable unit dosage quantity of the composition, it is preferred that the sealing sheet 18 is attached by means other than laminating, heat sealing or means that would otherwise not be readily available to a pharmacist or chemist. In such circumstances, flexible freezable self-sealing polymeric sheets, adhesives, mechanical means such as the interlock, tongue-in-groove, frictional or lid means described aforesaid or other similar means are preferred. It is also preferred that the adhesive does not come into significant contact with the unit dosage quantity upon sealing.

FIG. 4 illustrates an unsealed portion 15 near the edge of a cavity 14. This unsealed portion 15 allows a consumer to peel back the sealing sheet 18 from the film sheet 12 to uncover and remove a frozen unit dosage 16 contained in a cavity 14. The sealing sheet 18 can be made from any variety of materials such as polymer materials, polyester, metal foil, aluminum foil, cellulose, paper, combinations thereof and the like. In one embodiment, a sealing sheet 18 is preferably ruptureable upon manual compression of a cavity 14 containing a unit dosage 16 by a consumer. Thus, a storage container 10 can be re-inserted into a freezer after removing a unit dosage 16 such that any melted unit dosages 16 remaining in the container 10 can be re-freezed.

In one embodiment, as shown in FIG. 2, the sealing sheet 18 can comprise a single layer. Alternatively, as shown in FIG. 3, the sealing sheet 18 can comprise multiple layers 18a, 18b, 18c in a stacked arrangement with one another. For example, the sealing sheet 18 can comprise a stacked arrangement of a polymer layer 18a, an aluminum layer 18b and a paper layer 18c, wherein the polymer layer 18a is in contact with the unit dosage 16. In this stacked arrangement, the polymer layer 18a can provide strength and prevent inadvertent tearing of the aluminum foil layer 18b. The aluminum foil layer 18b acts as the primary barrier for safeguarding a unit dosage 16 being held within a cavity 14. The paper layer 18c can provide further strength to the sealing sheet 18. The paper layer 18c is preferably arranged away from the cavities 14 and creates the bottom face of a storage container 10. As such, a paper layer 18c can be suitable for printing and thereby allows for visual indicia, such as instructions for dosing and administration or information about the unit dosages 16, to be printed on the bottom face of a storage container 10. Other layers can also be suitable for printing visual indicia. For example, visual indicia can be printed on the polymer layer 18a or film sheet 12. Visual indicia can comprise labeling stating that the unit dosages 16 are to be administered orally in frozen form and that the product is clinically proven to reduce the severity and duration of cold and flu symptoms.

Unit dosages 16 can be removed from a storage container 10 by applying pressure to the cavity or depression 14 to force the frozen unit dosage 16 to rupture and pass through the sealing sheet 18. Perforations 20 can be made in between the cavities 14 of a storage container 10 to allow the separation of one or more unit dosage 16 from the film sheet 12. As shown in FIG. 1, perforations 20 may be made in the horizontal and/or vertical direction between the cavities 14. The perforations 20 preferably extend through the film sheet 12 and sealing sheet 18 such that the consumer may tear the desired number of unit dosages 16 from the storage container 10. A single unit dosage 16 of a frozen composition of the present invention is shown in FIG. 4. Unit dosages 16 can be removed from a storage container 10 by tearing along an outer edge near a line of perforations 20. Once a unit dosage 16 or multiple unit dosages 16 are separated from a container 10, peeling an unsealed portion 15 of a sealing sheet 18 away from the corner of a film sheet 12 may reveal the unit dosage 16 for use.

The compositions of the present invention may be aseptically filled and sterilized into a storage container 10 in liquid or flowable form. While sterilization may be accomplished before packaging, sterilization of the liquid product may also be conveniently accomplished following the packaging thereof, for example, the storage container 10 can be terminally sterilized. Sterilization may be accomplished by methods known in the art, such as by heating. The sterile compositions may be used following freezing thereof.

The unitary dosages provided by the invention may be more economical because there may be less product waste. The storage containers 10 containing the unit dosages 16 of the compositions of the present invention may be readily transported, and stored at room temperature or in the freezer for future use. In use, storage containers 10 are frozen, the blisters opened, and the unit dosages 16 administered orally, rectally or vaginally, as the case may be, to a human.

The compositions of the present invention may comprise water, solvent, plant-based or herbal ingredients, active ingredients, nutrient supplements including vitamins or minerals, carriers, binders, buffering agents, surfactants, preservatives, coloring agents, stabilizers, flavoring agents and sweeteners, or combinations thereof. The constituent ingredients and amounts of these ingredients may be adjusted to enhance the texture, stability, and freezing qualities, such as the freezing point, of the compositions. Preferably, the constituents and amounts contained in each unit dosage results in a composition that is stable in liquid or flowable form at room temperature and in solid frozen form at or below the freezing point of the composition.

The above ingredients may be selected and combined to enhance the delivery of at least one plant-based or herbal ingredient or a pharmaceutical active ingredient that treats the symptoms or conditions discussed herein. The above ingredients are preferably combined with at least one plant-based or herbal ingredient or a pharmaceutical active ingredient such that the resulting composition has a freezing point below 5° C., preferably below 3° C., or preferably substantially similar to that of water, i.e., 0° C., thus allowing conventional freezing equipment and techniques to be used. As will be appreciated, the freezing point of the composition, and particularly unit dosages thereof, may be lower than 0° C., for example, −5° C., −10° C., −15° C. or even −20° C. and still be amenable to freezing through the use of conventional freezing equipment and techniques, for example, a residential home freezer where such a freezer achieves such temperatures.

Preferably, the compositions of the present invention have a pH ranging from about 5 to about 6. As discussed herein, the pH of the present composition is measured by any suitable means well known to persons of ordinary skill in the art.

The freezable lozenge preferably is a buffered formulation. A preferred formulation is at a pH of about 6-11, and preferably at a pH of about 7-9. Preferred buffered formulations will include sodium carbonate, sodium bicarbonate, sodium phosphate, calcium carbonate, magnesium hydroxide, potassium hydroxide, magnesium carbonate, aluminum hydroxide, and other substances known to those skilled in the art, as well as any combination of the aforementioned substances. In a most preferred formulation, the freezable lozenge will contain sodium carbonate and bicarbonate as buffering agents.

The buffering agent(s) should be present in an amount sufficient to adjust the pH of the freezable lozenge to between 6 and 11, typically, between about 0.1 and 25% by weight (wt %), preferably in an amount between about 0.1 and 10 wt %, and more preferably in an amount between about 0.1 and 5 wt %.

Plant-based or herbal ingredients for use in compositions of the present invention can comprise, but are not limited to, garlic, arabinogalactan, St. John's wart, goldenseal, eyebright, licorice, rose hips, chamomile, slippery elm bark, echinacea, ginkgo biloba, red doer, sarsaparilla, uva ursi, aloe vera, black cohosh, green tea, cat's claw, devil's claw, buchu, ginseng, dandelion, raspberry leaf, spearmint, peppermint, comfrey, senna, feverfew, ginger, calendula, dong quai, cranberry, ephedra, hawthorn, juniper, thistle, valerian, elder tree, green coffee bean, green cacao bean, artichoke, birch tree, wild yam, agnus castus, passion flower, saw palmetto and schizandra. It is believed, without being bound thereto, that the herbal ingredient of the compositions of the present invention can treat or suppress the symptoms associated with a local oral or systemic condition. Additionally, a combination or some or all of these herbal ingredients, with or with other active ingredients, can be effective in treating or suppressing the symptoms or conditions discussed above.

Active ingredients, including pharmaceutical active ingredients, for use in compositions of the present invention can comprise, but are not limited to, dextromethophan, guaifenesin, lidocaine/sylocalne, phenylephrine, codeine sulfate, benzydamine, aspirin, non-steroidal anti-inflammatory drugs such as arylalkanoic acids, 2-arylpropionic acids, N-arylanthranilic acids, pryrazolidine derivatives, oxicans, COX-2 inhibitors or sulphonanilides, acetominophen, ibuprofen, other analgesics, antiseptics, antibiotics including amoxicillin, antifungals, antivirals, anti-infectives, decongestants, antihistamines, sedatives, anxiolytics, diuretics, stimulants, muscle relaxants, antacids, antipsycotics, antidepressants, neuroleptics, antipyretics, antitussives, steroids, chemotherapies, antiarrhythmics, diuretics, antinausea, hyperglycemics, antiepileptics, motility agents, antihypertensives or the like. As will be appreciated, this list is illustrative and not exhaustive.

As will also be appreciated, certain active ingredients will not totally dissolve but may form a suspension or be dispersed into a suitable liquid or flowable carrier. Certain active ingredients may also be formulated and encapsulated such that the active ingredient may be suspended in the frozen medium and released upon thawing. The addition of other agents to facilitate dissolution, suspension, dispersion, or the like, of one or more selected constituent ingredients is also contemplated.

The active agents may be available over the counter or the type prescribed. One or more active agents may be included in compositions of the present invention

Solvents for use in the compositions of the present invention comprise, but are not limited to, water, an alcohol, a polyol, a polyether polyol, a derivative of a polyether polyol, glycerol, gelatin, mineral oil, olive oil, corn oil or any other consumable biologic or synthetic oil.

Nutrient supplements for use in the compositions of the present invention comprise, but are not limited to, vitamin A, vitamin C, vitamin D, vitamin E, vitamin B1, vitamin B6, vitamin B12, niacinamide, pantothenic acid, thiamin, riboflavin, palm, choline inositol folic acid, biotin, calcium, magnesium, iron, chromium, manganese, zinc, potassium, phosphorus, selenium and iodine.

The preservatives for use in the compositions of the present invention comprise, but are not limited to, sodium benzoate and potassium benzoate. Although not required, preservatives are comprised in the compositions to extend the shelf life of the compositions and to prevent spoilage. Persons knowledgeable in the art would be able to select the appropriate preservative, in the proper amount, to accomplish this result.

Coloring agents for use in the compositions of the present invention comprise, but are not limited to, any of the USFDA certified food colors. Coloring agents can also comprise pigments such as titanium dioxide. Although not required, coloring agents can be comprised in the compositions to enhance the aesthetic appearance of the product and be used in an amount effective to produce a desired color, for instance when it can be view in a transparent blister pack. Coloring agents can encourage the consumer to associate a color with the flavor of the compositions, which can promote patient compliance.

As used herein the term “flavoring agent” comprises both fruit and botanical flavoring agents. A flavoring agent can be comprised in any of the compositions made according to this invention. Flavoring agents can comprise, but are not limited to, one or more natural and/or synthetic flavors, juice and/or oils derived from plants, leaves, flowers and fruit. For example, such flavors and oils of these types comprise acids such as adipic, succinic and fumaric acid; citrus oils such as lemon oil, orange oil, lime oil and grapefruit oil; fruit essences or extracts, such as apple essence, lemon essence, pear essence, peach essence, strawberry essence, apricot essence, raspberry essence, cherry essence, plum essence and pineapple essence; essential oils such as peppermint oil, spearmint oil, bay oil, anise oil, oil of nutmeg, oil of sage, oil of bitter almonds, cassia oil and methylsalicylate (oil of wintergreen). optional flavoring agents including peppermint, peppermint-menthol, eucalyptol, wintergreen, licorice, clove, cinnamon, spearmint, menthol and various combinations thereof. Although not required, without a flavoring agent, the consumer might want to swallow the unmelted, frozen composition to avoid the taste of other ingredients and before such ingredients have a chance to coat the oral cavity and absorb into the bloodstream of the consumer, such as through the consumer's mucosal and sub-lingual membranes.

As used herein the term “sweetener” comprises starch, partially hydrolyzed starch, sugars, for example, monosaccharides, disaccharides, polysaccharides, glucose, sucrose, lactose, maltose, dextrose and fructose. Sugars also comprise honey, maltodextrins, high fructose corn syrup solids, invert sugar, sugar alcohols including sorbitol, xylitol, mannitol, and mixtures thereof. Artificial sweeteners are also comprised within the scope of the term, “sweetener,” for example, soluble saccharin salts, nutrasweet, sucralose, acesulfane-K, etc. A sweetener can be comprised in any of the compositions made according to this invention.

The compositions of the present invention can be manufactured or formulated using techniques well known to those skilled in the art, including by blending, mixing, dispersing, emulsifying, and the like. Agitation or heating to the appropriate temperature to dissolve all the constituents or ingredients, may also be employed, if desired. The compositions may be packaged into storage containers and sterilized to food grade standards as is known in the art. Packaging of the compositions into storage containers is further described above.

### EXAMPLES

The following examples are illustrative of preferred embodiments of the invention and are not to be construed as limiting the invention thereto. All percentages are based on the percent by weight of the composition unless otherwise indicated and all totals equal 100% by weight.

### Example 1

A lozenge-forming composition may be a sugar-based, sugar alcohol-based, water-based or sugar water-based composition. A lozenge-forming composition that is sugar-based or sugar water based may comprise a single sugar (e.g. sucrose) or a mixture of sugars (e.g. a mixture of sucrose and glucose) or it may comprise sorbitol, xylitol, malitol, malitol syrup, lactitol, mannitol or mixtures thereof which may be in the form of the free sugar alcohols, derivatives thereof or mixtures thereof. In addition to the components listed above, the freezable lozenge formulations provided by the present invention may contain other ingredients such as acidity regulators, opacifiers, stabilizing agents, buffering agents, flavourings, sweeteners, colouring agents and preservatives.

It may also include, but is not limited to phenol, menthol, sodium phenolate, benzocaine, and cetylpyridinium chloride or any combination of analgesics, anesthetics, antiseptics, antimicrobials, antitussives, anti-nauseants, mucloytics and decongestants. It may also include, but is not limited to any of the herbal medication listed above.

### Example 2

### Amoxicillin

### Amoxicillin Lozenge

Each lozenge of approximately 5 mL volume may contain:
50 mg of Amoxicillin Sodium Powder carried in a polyglycol base.

### Silica Gel Powder 1%

Flavouring (3% weight of lozenge)—for example, Raspberry or Cherry flavour together with an additional 3% Marshmallow flavour to combat bitterness

### Water

As will be appreciated by persons skilled in the art, proportions and constituents may be varied, as dependant upon such factors as aesthetic appearance, pharmaceutical elegance, and end-use palatability.

To increase stability and extend expiry, it is preferred that the liquid vehicle base and the amoxicillin powder be separately stored for mixing prior to use, if extended shelf life is desirable. The constituents may be mixed by shaking, added in 5 mL increments into storage container cavities and frozen for use.

### Example 3

### Acetominophen

Acetominophen lozenge may be prepared as above, substituting 50 mg of Acetaminophen for Amoxicillin Sodium.

Grape flavour (3% by weight) or Tutti-frutti (3%) may be desired to mask taste. Additional flavouring such a 3% marshmallow, may be desirable.

While the invention has been described with reference to preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will comprise all embodiments falling within the scope of the appended claims.

What is claimed is:

1. A freezable unit dosage delivery system comprising:
(a) a composition comprising at least one active ingredient and a physiologically or pharmaceutically acceptable carrier, said composition having a freezing point of 5° C. or less;
(b) a storage container comprising one or more liquid-impermeable cavities, each cavity configured to receive a unit dosage quantity of the composition; and
(c) a sealing sheet for sealing engagement with the storage container, the sealing sheet adapted to cooperate with at least one cavity to define a unit dosage of the composition and to form a leak resistant seal enclosing the at least one cavity suitable for containing the unit dosage quantity of the composition within the cavity;
wherein the unit dosage quantity solidifies into a solid unit dosage of the composition within each cavity upon freezing.

2. The freezable unit dosage delivery system of claim 1 wherein unit dosage quantity is in liquid or flowable form at room temperature prior to freezing.

3. The freezable unit dosage delivery system of claim 1 wherein the unit dosage quantity is in liquid or flowable form at a temperature in the range of 10° C. to 35° C. prior to freezing.

4. The freezable unit dosage delivery system of claim 1 wherein the composition has a freezing point below 0° C.

5. The freezable unit dosage delivery system of claim 1 wherein the unit dosage quantity solidifies into a solid unit dosage form upon freezing at a temperature in the range of −20° C. to 5° C.

6. The freezable unit dosage delivery system of claim 1 wherein the solid unit dosage substantially conforms to the shape defined by the cavity.

7. The freezable unit dosage delivery system of claim 1 wherein the solid unit dosage is suitable for one of oral, rectal or vaginal administration, for locally or systemically treating symptoms or conditions of a human.

8. The freezable unit dosage delivery system of claim 1 wherein the solid unit dosage is in a configuration selected from the group consisting of lozenge, tablet, capsule, ring, star, geometric shapes, suppository, torpedo or candy confectionary.

9. The freezable unit dosage delivery system of claim 1 wherein the storage container and sealing sheet are in the form of a blister pack.

10. The freezable unit dosage delivery system of claim 1 wherein the sealing sheet is adapted to releasably sealing engage the storage container.

11. The freezable unit dosage delivery system of claim 1 wherein the sealing sheet and the storage container are further provided with sealing means adapted to sealingly engage the sealing sheet to the storage container on manual application.

12. The freezable unit dosage delivery system of claim 1 wherein the active ingredient is an herbal, pharmaceutical or nutritional ingredient.

13. The freezable unit dosage delivery system of claim 12 wherein the active ingredient is selected from the group consisting of antibiotic agents, antifungal agents, antiinfective agents, antiviral agents, antiseptics, analgesics, decongestants, antihistamine, sedatives, anxiolytics, diuretics, stimulants, expectorants, muscle relaxants, antacids, anti-inflammatories, antipsycotics, antidepressants, neuroleptics, antipyretics, antitussives, steroids, chemotherapies, antiarrhythmics, diuretics, antinausea, hyperglycemics, antiepileptics, motility agents, and antihypertensives.

14. The freezable unit dosage delivery system of claim 12 wherein the active ingredients selected from the group consisting of dextromethophan, guaifenesin, lidocaine/sylocalne, phenylephrine, codeine sulfate, benzydamine, aspirin, non-steroidal anti-inflammatory drugs such as arylalkanoic acids, 2-arylpropionic acids, N-arylanthranilic acids, pryrazolidine derivatives, oxicans, COX-2 inhibitors or sulphonanilides, acetominophen, ibuprofen and amoxicillin.

15. The freezable unit dosage delivery system of claim 12 wherein the active ingredient is selected from the group consisting of garlic, arabinogalactan, St. John's wart, goldenseal, eyebright, licorice, rose hips, chamomile, slippery elm bark, echinacea, ginkgo biloba, red doer, sarsaparilla, uva ursi, aloe vera, black cohosh, green tea, cat's claw, devil's claw, buchu, ginseng, dandelion, raspberry leaf, spearmint, peppermint, comfrey, senna, feverfew, ginger, calendula, dong quai, cranberry, ephedra, hawthorn, juniper, thistle, valerian, elder tree, green coffee bean, green cacao bean, artichoke, birch tree, wild yam, agnus castus, passion flower, saw palmetto and schizandra.

16. The freezable unit dosage delivery system of claim 12 wherein the active ingredient is selected from the group consisting of vitamin A, vitamin C, vitamin D, vitamin E, vitamin B1, vitamin B6, vitamin B12, niacinamide, pantothenic acid, thiamin, riboflavin, paba, choline inositol folic acid, biotin, calcium, magnesium, iron, chromium, manganese, zinc, potassium, phosphorus, selenium and iodine.

17. The freezable unit dosage delivery system of claim 1 wherein the physiological or pharmaceutically acceptable carrier includes water, an alcohol, a polyol, a polyether polyol, a derivative of a polyether polyol, glycerol, gelatin, mineral oil, olive oil, corn oil or any other consumable biologic or synthetic oil.

18. The freezable unit dosage delivery system of claim 1 wherein the composition further includes one or more constituents selected from the group consisting of buffering agents, stabilizers, surfactants, preservatives, coloring agents, sweeteners, or flavouring agents.

19. The freezable unit dosage delivery system of claim 1 wherein the sealing sheet is adapted for sealing engagement with the storage container by means selected from the group consisting of adhesive, heat sealing, lamination, or mechanical means, including lid means, frictional engagement means, tongue-in-groove means and interlocking means.

20. A method for preparing a freezable unit dosage, comprising the steps of:
(a) providing a composition comprising at least one active ingredient and a physiologically or pharmaceutically acceptable carrier, said composition has a freezing point of about 5° C. or less;
(b) providing a storage container comprising one or more liquid-impermeable cavities, each cavity configured to receive a unit dosage quantity of the composition,
(c) placing a unit dosage quantity of the composition into at least one or more of the cavities; and
(d) applying a sealing sheet to sealing engage the storage container, the sealing sheet cooperating with at least one cavity to define a unit dosage of the composition and forming a leak resistant seal enclosing the unit dosage quantity of the composition within the cavity,
wherein the unit dosage quantity solidifies into a solid unit dosage of the composition within each cavity upon freezing.

21. The method of claim 20 further providing instructions to subject the storage container containing the unit dosages to freezing.

22. The method of claim 21 wherein freezing includes subjecting the storage container to a temperature of below 5° C., for a period of time sufficient to freeze the unit dosages into a solid unit dosage form.

23. The method of claim 21 wherein freezing includes subjecting the storage container to a temperature of below 0° C., for a period of time sufficient to freeze the unit dosages into a solid unit dosage form.

24. The method of claim 21 wherein freezing includes subjecting the storage container to a temperature in the range of −20° C. to 0° C., for a period of time sufficient to freeze the unit dosages into a solid unit dosage form.

25. The method of claim 21 wherein unit dosage quantity is in liquid or flowable form at room temperature prior to freezing.

26. The method of claim 21 wherein the unit dosage quantity is in liquid or flowable form at a temperature in the range of 10° C. to 35° C.

27. The method of claim 21 wherein the composition has a freezing point below 0° C.

28. The method of claim 21 wherein the unit dosage quantity solidifies into a solid unit dosage form upon freezing at a temperature in the range of −20° C. to 5° C.

29. The method of claim 21 wherein the sealing sheet is manually applied to the storage container.

30. The method of claim 21 wherein the sealing sheet is applied using an adhesive or mechanical means.

31. The method of claim 21 wherein the active ingredient is an herbal, pharmaceutical or nutritional ingredient.

32. The method of claim 21 wherein the physiological or pharmaceutically acceptable carrier includes water, an alcohol, a polyol, a polyether polyol, a derivative of a polyether polyol, glycerol, gelatin, mineral oil, olive oil, corn oil or any other consumable biologic or synthetic oil.

33. The method of claim 21 wherein the one or more cavities of the storage container are selected so as to define a configuration for the solid unit dosage subsequent to freezing.

34. A solid unit dosage form prepared by the method according to any one of claims 20 to 33.

35. The solid unit dosage form of claim 34, wherein the solid unit dosage form is adapted for one of oral, vaginal or rectal administration, for locally or systemically treating symptoms or conditions of a human.

36. Use of a solid unit dosage form prepared by the method according to any one of claims 20 to 33 for locally or systemically treating symptoms or conditions of a human.

37. A method for locally or systemically treating symptoms or conditions of a human in need of such treatment comprising the steps of:
(a) providing a solid unit dosage prepared by:
(i) providing a composition comprising at least one active ingredient and a physiologically or pharmaceutically acceptable carrier, said composition has a freezing point of about 5° C. or less;
(ii) providing a storage container comprising one or more liquid-impermeable cavities, each cavity configured to receive a unit dosage quantity of the composition,
(iii) placing a unit dosage quantity of the composition into at least one or more of the cavities; and
(iv) applying a sealing sheet to sealing engage the storage container, the sealing sheet cooperating with at least one cavity to define a unit dosage of the composition and forming a leak resistant seal enclosing the unit dosage quantity of the composition within the cavity;
(b) subjecting the storage container containing the unit dosages to freezing;
(c) removing at least one of the solid unit dosages from said storage container; and
(d) administering said at least one solid unit dosage to a human.

38. The method of claim 37 wherein freezing includes subjecting the storage container to a temperature of below 5° C., for a period of time sufficient to freeze the unit dosages into a solid unit dosage form, said solid unit dosage form suitable for one of oral, rectal or vaginal administration.

39. The method of claim 38 wherein freezing includes subjecting the storage container to a temperature of below 0° C., for a period of time sufficient to freeze the unit dosages into a solid unit dosage form.

40. The method of claim 38 wherein freezing includes subjecting the storage container to a temperature in the range of −20° C. to 0° C., for a period of time sufficient to freeze the unit dosages into a solid unit dosage form.

41. The method of claim 37 wherein unit dosage quantity is in liquid or flowable form at room temperature prior to freezing.

42. The method of claim 37 wherein the unit dosage quantity is in liquid or flowable form at a temperature in the range of 10° C. to 35° C., prior to freezing.

43. The method of claim 37 wherein the composition has a freezing point below 0° C.

44. The method of claim 37 wherein the unit dosage quantity solidifies into a solid unit dosage form upon freezing with a temperature in the range of −20° C. to 5° C.

45. The method of claim 37 wherein the symptoms and conditions is one or more of bacterial, viral, and fungal infections, the common cold or flu, bronchitis, upper respiratory tract infection, pneumonia, aches, pain, headache, nasal and sinus congestion, sore throat, cough, hoarseness, oral or rectal discomfort and irritation, malaise, fatigue, insomnia, mood disorders, depression, cholesterol level disorders, allergic reactions, stress, sleeplessness, anxiety, digestive disorders, gastrointestinal disorders, kidney disorders, bowel and urinary disorders, dementia, delirium, fever, hypertension, heart and vascular disorders, diabetes and related conditions, inflammation, autoimmune and immunodeficiency disorders, respiratory disorders, and disorders due injury.

46. A kit comprising:
(a) a composition comprising at least one active ingredient and a physiologically or pharmaceutically acceptable carrier, said composition having a freezing point of 5° C. or less;
(b) a storage container comprising one or more liquid-impermeable cavities, each cavity configured to receive a unit dosage quantity of the composition; and
(c) a sealing sheet for sealing engagement with the storage container, the sealing sheet adapted to cooperate with at least one cavity to define a unit dosage of the composition and to form a leak resistant seal enclosing the unit dosage quantity of the composition within the cavity; and
(d) an instruction to subject the storage container containing the unit dosages in at least one or more of the cavities to freezing;
wherein the unit dosage quantity solidifies into a solid unit dosage of the composition within each cavity upon freezing, said solid unit dosage form suitable for one of oral, rectal or vaginal administration.

47. The kit of claim 46 wherein freezing includes subjecting the storage container to a temperature of below 5° C., for a period of time sufficient to freeze the unit dosages into a solid unit dosage form, said solid unit dosage form suitable for one of oral, rectal or vaginal administration.

48. The kit of claim 46 wherein freezing includes subjecting the storage container to a temperature of below 0° C., for a period of time sufficient to freeze the unit dosages into a solid unit dosage form.

49. The kit of claim 46 wherein freezing includes subjecting the storage container to a temperature in the range of −20° C. to 0° C., for a period of time sufficient to freeze the unit dosages into a solid unit dosage form.

50. The kit of claim 46 wherein the instruction further comprises an instruction to administer the at least one of the solid unit dosages.

51. A solid unit dosage for one of oral, vaginal or rectal administration for locally or systemically treating symptoms or conditions of a human in need of such treatment, comprising a unit dosage quantity of a composition, said composition comprising at least one active ingredient and a physiologically or pharmaceutically acceptable carrier, said composition having a freezing point of 5° C. or less; said unit dosage quantity being in liquid or flowable form at room temperature prior to freezing, and said unit dosage quantity solidifying into a solid unit dosage upon freezing,

52. The solid unit dosage of claim 51 wherein the unit dosage quantity is in liquid or flowable form at a temperature in the range of 10° C. to 35° C. prior to freezing.

53. The solid unit dosage of claim 51 wherein the unit dosage quantity solidifies into a solid unit dosage form upon freezing at a temperature in the range of −20° C. to 5° C.

54. The solid unit dosage of claim 51 wherein the unit dosage quantity substantially conforms to the configuration defined by a storage container or a compartment thereof within which the unit dosage quantity is contained when subjected to freezing, thereby providing a configuration for the solid unit dosage.

55. The solid unit dosage of claim 51 wherein the solid unit dosage is adapted for one of oral, rectal or vaginal administration.

56. The solid unit dosage of claim 54 wherein the solid unit dosage is in a configuration selected from the group consisting of lozenge, tablet, capsule, ring, star, geometric shapes, suppository, torpedo or candy confectionery.

57. The solid unit dosage of claim 51 wherein the active ingredient is an herbal, pharmaceutical or nutritional ingredient.

58. The solid unit dosage of claim 51 wherein the active ingredient is selected from the group consisting of dextromethophan, guaifenesin, lidocaine/sylocalne, phenylephrine, codeine sulfate, benzydamine, aspirin, non-steroidal anti-inflammatory drugs such as arylalkanoic acids, 2-arylpropionic acids, N-arylanthranilic acids, pryrazolidine derivatives, oxicans, COX-2 inhibitors or sulphonanilides, acetominophen, ibuprofen, amoxicillin, garlic, arabinogalactan, St. John's wart, goldenseal, eyebright, licorice, rose hips, chamomile, slippery elm bark, echinacea, ginkgo biloba, red doer, sarsaparilla, uva ursi, aloe vera, black cohosh, green tea, cat's claw, devil's claw, buchu, ginseng, dandelion, raspberry leaf, spearmint, peppermint, comfrey, senna, feverfew, ginger, calendula, dong quai, cranberry, ephedra, hawthorn, juniper, thistle, valerian, elder tree, green coffee bean, green cacao bean, artichoke, birch tree, wild yam, agnus castus, passion flower, saw palmetto and schizandra.

59. The solid unit dosage of claim 51 wherein the active ingredient includes one of acetaminophen, amoxicillin, ginseng or Echinacea.

60. The solid unit dosage according to any one of claim 7, 35, 36 or 51 wherein the symptoms and conditions is one or more of bacterial, viral, and fungal infections, the common cold or flu, bronchitis, upper respiratory tract infection, pneumonia, aches, pain, headache, nasal and sinus congestion, sore throat, cough, hoarseness, oral or rectal discomfort and irritation, malaise, fatigue, insomnia, mood disorders, depression, cholesterol level disorders, allergic reactions, stress, sleeplessness, anxiety, digestive disorders, gastrointestinal disorders, kidney disorders, bowel and urinary disorders, dementia, delirium, fever, hypertension, heart and vascular disorders, diabetes and related conditions, inflammation, autoimmune and immunodeficiency disorders, respiratory disorders, and disorders due injury.

61. The solid unit dosage of claim 60 wherein the solid unit dosage is a throat lozenge.

62. The solid unit dosage of claim 60 wherein the solid unit dosage is a rectal or vaginal suppository.
